(19) United States
(12) Patent Application Publication
(10) Pub. No.: US 2019/0008761 A1
(43) Pub. Date: Jan. 10, 2019

(54) METHOD TO FORMULATE AND PRODUCE DIETARY SUPPLEMENTS OR FUNCTIONAL FOODS IN EFFERVESCENT SPHEROIDAL SHAPED TABLETS

(71) Applicant: DRAGOS ALEVIZACHE, MCKINNEY, TX (US)

(72) Inventor: DRAGOS ALEVIZACHE, MCKINNEY, TX (US)

(21) Appl. No.: 15/643,502

(22) Filed: Jul. 7, 2017

(51) Int. Cl. A61K 9/46; A61K 9/20; A61K 31/197; A23L 33/10; A23L 33/00; A23P 10/28

(52) U.S. Cl. CPC A61K 9/0007; A61K 9/2095; A61K 31/197; A23V 2002/00; A23L 33/40; A23P 10/28; A23L 33/10

(57) ABSTRACT

This application discloses effervescent spheroidal shaped tablets for dietary supplements, nutraceuticals, or functional foods, and methods for making and producing the tablets.

### FIELD OF INVENTION

The present disclosure generally relates to novel forms of representation of dietary supplement formulations used by general wellness consumers, professional athletes or bodybuilders, such as pre-training, intra-training or post training programs. The tablets can also take other shapes, such as parallelepiped, cylinders or combined shapes, which may include, for example a sphere attached to a parallelepiped, and single or multilayer formulations.

### BACKGROUND

In the current market of dietary supplements or foods, and especially the more specialized area of sports dietary supplements, there is an inconvenience of current forms of representation; the inconvenience start from the big serving size of effective dosages of amino acids or other ingredients used as dietary ingredients in formulations. The effective dosage of such ingredients usually at the level of few grams per serving is making the vast majority form of current representations to be a powder mix, swollen tablets, capsules or soft gel capsules. The tablets and capsules are limited to traditional tablets size or capsules sizes from few milligrams to 1-3 grams per piece.

The powder mixes for drink preparations are packaged into jars of different shapes and sizes and have a scoop inside the container. The consumer should tear the induction seal, and use fingers to find the scoop inside every time is a need of usage. This make it inconvenient and non-sanitary, the powders mix generally heaving a fluffy, hygroscopic or static nature.

The capsules and tablets are heaving the inconvenient that are only on swallow able or chewable forms and require the consumer to take usually more than 1 and sometimes even 10 capsules or tablets to satisfy the effective dosage. The chewable have also the inconvenient to be sometimes hard to chew or are made of a base that make the stomach upset such as sugar alcohols.

Effervescent tablets or granules are uncoated and generally contain acidic substances and carbonate or bicarbonates which react rapidly to release carbon dioxide when dissolve in water. There are various advantages of effervescent formulations such as fast onset of action, good stomach tolerance, improves palatability, enhance permeability, but major problem which is associated with these formulations is their sodium and potassium content which is present in the form of carbonate/bicarbonates.

According to the current guidance the per day sodium intake is advised to be limited to 2400 mg and the potassium to 4,700 mg. This excess of sodium and potassium will produce heath complications particular to cardiac and renal patients. Patent number CA 2706353 C, describe the manufacturing process of arginine bicarbonate salt.

The science of effervescence is a dynamic field of study. The chemical reactions producing carbon dioxide (CO₂) allows our bodies to absorb dietary supplements and/or pharmaceuticals more effectively. CO₂ created by the effervescent reaction induces “enhanced active-ingredient permeability” due to an alteration of the para-cellular pathway.

Traditional effervescent tablets constitute various ingredients and are dissolved in liquids, to be absorbed quickly, completely and uniformly by the body. A high quality well-formulated effervescent product entering the stomach, where the ingredients are already evenly distributed in the solution, and highly localized concentration of the compounds cannot occur.

State of the art teaches a fast-dissolving effervescent tablet requires a large surface area in order to dissolve quickly in water. It has long been held that the most efficient way to ensure quick dissolution, was to make tablets in the classic disk shape.

Pharmaceutical companies that pioneered the concepts of the effervescent tablets designed the effervescent tablets for small dosages where only one or two active ingredients were targeted to be delivered. On the other hand, the nutraceuticals or dietary supplement industry formulations attempt to deliver multiple dietary supplements in one dose to achieve effective dosages of individual ingredients. This presents a challenge, as a bigger tablet is inconvenient to produce, slowing down the production, increasing production costs, and becoming inconvenient for the consumers to take.

The embodiments, formulations and tablets described herein use a novel approach to solve the current need in the nutraceutical or dietary supplement field. One advantage of the spheroidal shaped tablet versus classical disc shaped tablet is better dissolution times in water or other liquids when compare with the amount of active ingredients per tablet versus excipients levels. Surprisingly, and contrary to state of art, the effervescent spheroidal shaped tablets described herein release CO₂ bubble gas formed from the reaction with water in less time than a traditional disc shaped effervescent tablet due to the surface tension created between the gas and the tablet. In many cases, effervescent spheroidal shaped tablets described herein obtain comparable dissolution rates to traditional disc shaped effervescent tablet despite the fact that the effervescent spheroidal shaped tablets weight is higher.

### SUMMARY

The embodiments of the present invention may teach how to incorporate one or more dietary supplement ingredients, including but not limited to amino acids, herbs, herbal extracts, natural metabolites of the body, vitamins, or minerals, at effective dosage levels into one or more spheroidal shaped tablets, such that the tablets will disintegrate quickly in water, juice, milk, any beverage or other aqueous solution.

The embodiments of the present invention may teach how to formulate tablets as spheroidal shaped dietary supplements that achieve similar or better disintegration times than a similarly sized, dietary supplement tablet with typical big surface area, flat disc shape. These disintegration times may be achieved with or without the usage of carbonate or bicarbonate type of salts, including but not limited to sodium, potassium, calcium or any other pharmaceutically acceptable salt.

The embodiments described herein may also include dietary supplements of amino acid salts, carbonate salts, or bicarbonate salts, such as lysine bicarbonate, arginine bicarbonate. Such amino acids and bicarbonates may be used in combination with citric acid, malic acid, tartaric acid, fumaric acid, vitamin C (ascorbic acid) or ascorbates salts, alpha ketoglutaric acid in order to achieve the effervescent reaction when dissolved in water.

The term dietary ingredient as it is used herein includes any known dietary supplement, including but not limited to one or more combined dietary supplements as defined and described by U.S. 21 C.F.R. part 111 and 21 C.F.R. part 110.

The embodiments described herein may also achieve similar or better absorption of dietary ingredients in the body that are becoming more bioavailable by the alkaline nature of the media and the CO₂ gas released during the disintegration time in water.

The embodiments described herein may achieve similar or better disintegration rates of spheroidal shaped effervescent tablets when compared to the classic disc shaped effervescent tablets by nature of formulation and by the nature of the spheroidal shape, minimizing surface area that come in contact with the container with water in which the tablet will dissolve allowing the CO₂ gas bubbles formed during the reaction to be released faster.

Spheriodal as it is used in this disclosure may mean spherical, roughly spherical, capsule shaped, egg shaped or any other similar rounded three-dimensional shape;

One novel form of representation may be spheroidal shaped effervescent tablets with diameters between approximately 0.1″ to 3″ inches, and more preferably approximately 0.2″, 0.3″, 0.4″, 0.5″, 0.6″, 0.7″, 0.8″, 0.9″, 1.0″, 1.2″, 1.4″, 1.6″, 1.8″, 2.0″, 2.2″, 2.4″, 2.6″, 2.8″, and 3.0 inches in diameter.

An embodiment is a method of making an effervescent spheroidal tablet by the steps of: sifting one or more acids, one or more carbonates, and one or more dietary ingredients with a stainless steel sieve to create a mixture of components; mixing the mixture in a blender, and concurrently spraying mixing spray onto the mixture, until the mixture and mixing spray are homogenized; discharging the mixture onto a drying tray, drying the mixture in a preheated oven for a period of approximately 90 minutes, concurrently agitating the mixture; optionally allowing the mixture to cool to room temperature; and loading the mixture into a tablet press and operate tablet press to create a tablet.

An embodiment is a method of making an effervescent spheroidal tablet or formulation thereof by the steps of: sifting one or more acids, one or more carbonates, one or more dietary ingredients, one or more super-disintegrant agents, one or more binders, one or more lubricants, and one or more sweeteners with a stainless steel sieve to create a mixture of components; mixing the mixture in a blender, and concurrently spraying mixing spray onto the mixture, until the mixture and mixing spray are homogenized; discharging the mixture onto a drying tray; drying the mixture in a preheated oven for a period of approximately 90 minutes, concurrently agitating the mixture; optionally allowing the mixture to cool to room temperature; and loading the mixture into a tablet press and operate tablet press to create a tablet.

One or more dietary ingredients used in any method of making a spheroidal tablet or formulation thereof described herein, may be selected from a group consisting of caffeine anhydrous, tea extracts, purine alkaloids, theacrine, methyl liberine, 1,3,7 trimethyluric acid, 1,3,7 trimethyluric acid sodium salts, yohimbine, alpha yohimbine, yohimbine hydrochloride, huperzin A, vinitrox, creatine, creatine salts, buffered creatine, beta alanine, gamma butyrobetaine ethyl ester, gamma butyrobetaine ethyl ester hydrochloride, gammabutyrobetaine ethyl ester, medium chain triglycerides, carnitine, L-carnitine, fumarate, tartrate, paradoxine, dihydrocapsiate, branch chain amino acids, any mixture of one or more amino acids, leucine, isoleucine, valine, arginine base, alpha ketoglutarate hydrochloride, agmatine, agmatine salts, nitrosigine, B vitamins, and any pharmaceutical salts thereof.

Any blender used to make a spheroidal tablet or formulation thereof described herein, may be selected from group consisting of a ribbon blender, V-shaped blender, double-cone blender, any medium size, pilot size or any rotary industrial blender, spray drying blenders and fluidized bed equipment.

Any method of making a spheroidal tablet or formulation thereof may include a tablet or formulation having a stoichiometric ratio acid to carbonate, such that the one or more acids to the one or more carbonates is between 1:3 and 1:10.

Any method of making a spheroidal tablet or formulation thereof may include a tablet or formulation having a stoichiometric ratio acid to carbonate, such that the one or more acids to the one or more carbonates, derived the sodium bicarbonate and the sodium carbonate, allows the acid to quench the carbonate in an effervescent reaction, and after said effervescent reaction having an excess of the acid in an amount to achieve good tasting drinks after the tablet is dissolved in water completely.

Any method of making a spheroidal tablet or formulation thereof, which may use a stainless steel sieve, wherein the stainless steel sieve has a mesh size between 20 and 200, and more preferably the mesh size is between 40 and 80 mesh.

Any method of making a spheroidal tablet or formulation thereof, wherein the tablet or formulation may be made in an environment, and said environment has a relative humidity of less than 30%, and more preferably has a relative humidity between 10 and 20^{%}.

Any method of making a spheroidal tablet or formulation thereof, wherein the mixing spray may be comprised of water and the solid mass, and said mixing spray is 0.1% to 2% by weight of the total solid mass, wherein solid mass represent all the components in the formulation that were prior mixed or just the acid/bicarbonate/carbonate mixture);

Any method of making a spheroidal tablet or formulation thereof, The method of claim 11, wherein the mixing spray is 0.5% to 1% by weight of solid mass wherein solid mass represent all the components in the formulation that were prior mixed or just the acid/bicarbonate/carbonate mixture);

Any method of making a spheroidal tablet or formulation thereof, wherein the preheated oven is between 40 and 90 C, and more preferably wherein the preheated oven is between 70 and 90 C.

An embodiment of a spheroidal shaped effervescent tablet, wherein the spheroidal shaped effervescent tablet may have a diameter between 0.1″ to 3″, wherein said tablet comprises one or more dietary ingredients, one or more super-disintegrant agents, one or more binder, one or more lubricant, one or more acids, one or more carbonates, and one or more sweeteners, and wherein said tablet disintegrates as quickly as a disc shaped containing the same amount of active ingredients.

An embodiment of a spheroidal tablet, wherein the spheroidal tablet may be shaped like a capsule, having a first diameter between 0.25″ to 0.40″ and a second elongated diameter between 0.5″ and 1″.

An embodiment of a spheroidal tablet or formulation thereof, wherein the one or more dietary ingredients may be selected from any dietary supplement described in U.S. 21 C.F.R. part 111.

An embodiment of a spheroidal tablet or formulation thereof, wherein the one or more dietary ingredient may be selected from a group consisting of caffeine anhydrous, tea extracts, purine alkaloids, theacrine, methyl liberine, 1,3,7 trimethyluric acid, 1,3,7 trimethyluric acid sodium salts, yohimbine, alpha yohimbine, yohimbine hydrochloride, huperzin A, vinitrox, creatine, creatine salts, buffered creatine, beta alanine, gamma butyrobetaine ethyl ester, gamma butyrobetaine ethyl ester hydrochloride, gammabutyrobetaine ethyl ester, medium chain triglycerides, carnitine, L-carnitine, fumarate, tartrate, paradoxine, dihydrocapsiate, branch chain amino acids, any mixture of one or more amino acids, leucine, isoleucine, valine, arginine base, alpha ketoglutarate hydrochloride, agmatine, agmatine salts, nitrosigine, B vitamins, and any pharmaceutical salts thereof.

### BRIEF DESCRIPTION OF DRAWINGS

For more a complete understanding of this disclosure, reference is now made to the following description, taken in connection with the accompanying drawings as follows.

FIG. 1 depicts a graphic representation of the tablet press tooling used for the spheroidal shape effervescent dietary supplements tablets.

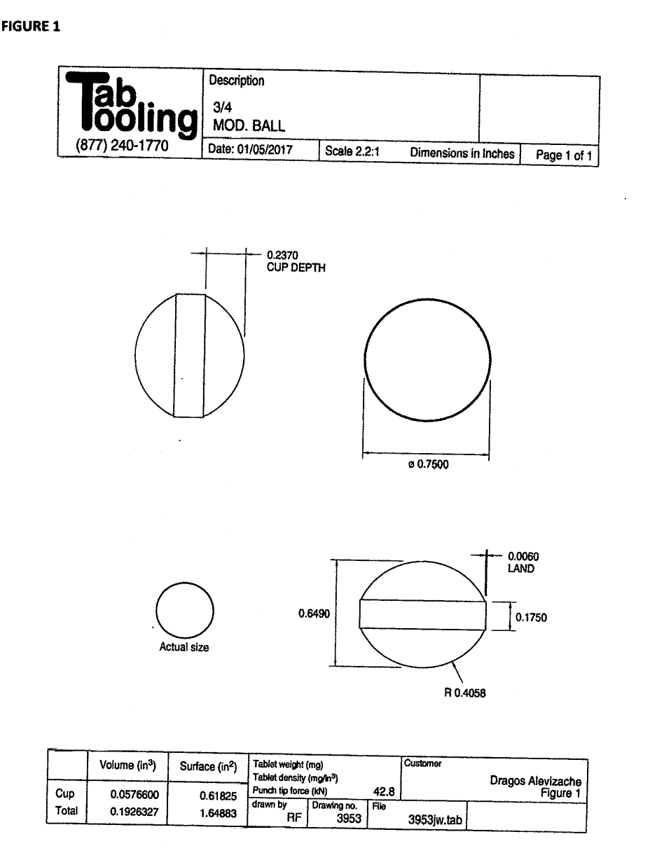

FIG. 2 depicts a graphic representation of the spheroidal shape tablet side by side with the classic disc effervescent tablet representation to describe the area of contact between the tablet surface and the bottom of a regular container to be dissolved within.

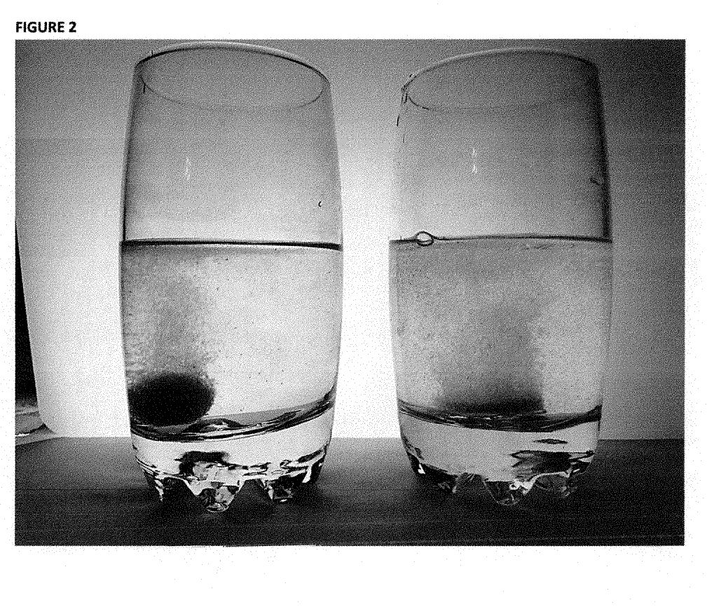

FIG. 3 depicts an image of a classic disc effervescent tablet and the spheroidal shaped tablet while dissolving.

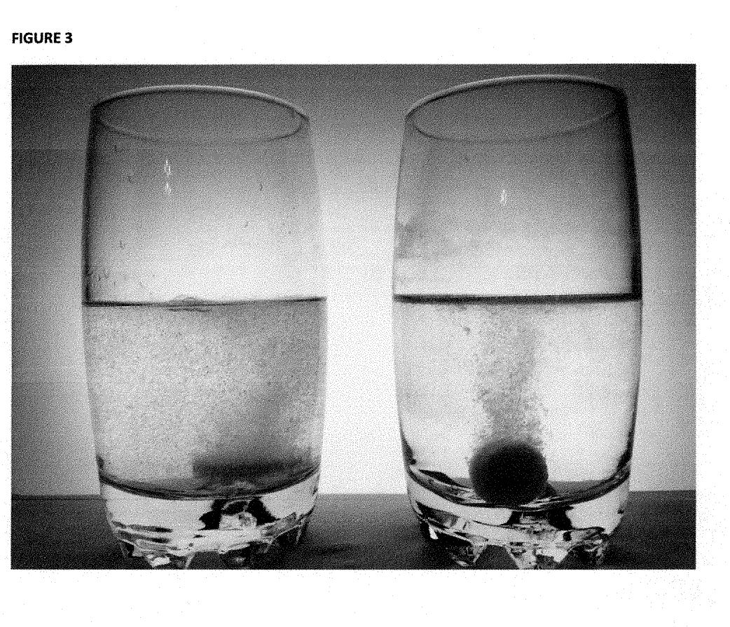

### DETAILED DESCRIPTION

This disclosure describes formulations for use in effervescent spheroidal shaped tablets, and methods of making same.

Any formulation may contain a dietary supplements known in the art including, but not limited to any amino acids, herbs, herbal extracts, vitamins, minerals, natural metabolites of the body, nutraceuticals, or any combination thereof; the supplements may be present in such formulations in any form of representation such as base, salts or derivatives, concentrates of natural plant extracts of such, peptides, enzymes, pro-biotics, carbohydrates, etc.

Any formulation may contain one or more dietary supplement, wherein said dietary supplement may be Caffeine Anhydrous, natural caffeine sources (including but not limited to tea extracts or related purine alkaloids such as Theacrine, Methyl Liberine, 1,3,7 trimethyluric acid, 1,3,7 Trimethyluric acid sodium salts), Yohimbine or Yohimbe from plant extracts or any pharmaceutically acceptable salt thereof, Alpha Yohimbine, Yohimbine HCL, Huperzin A, Arginine Base, Vinitrox (apple extract standardized for polyphenols) Creatine or creatine salts, Buffered Creatine forms, Beta Alanine, Gamma Butyrobetaine Ethyl Ester, Gamma Butyrobetaine Ethyl Ester or pharmaceutically acceptable salt thereof, Gamma Butyrobetaine Ethyl Ester Hydrochloride, Gammabutyrobetaine Ethyl Ester or pharmaceutically acceptable salt thereof, GBB EE HCL (Gamma Butyrobetaine Ethyl Ester Hydrochloride), MCT (Medium Chain triglycerides), Carnitine or L-Carnitine salts (including but not limited to as fumarate and tartrate), Paradoxine (6-Paradol), Dihydrocapsiate, BCAA (Branch Chain Amino Acids), any mixture of one or more amino acids, each amino acid having one or more aliphatic side chains with a central carbon bound to three or more carbon atoms), Leucine, Isoleucine, Valine, Arginine Base or Arginine salts such as alpha ketoglutarate o hydrochloride, agmatine or agmatine salts such as sulfate, hydrochloride or phosphate Arginine Inositol Silicate, B vitamins in any form (including but not limited to B1 (thiamine), B2 (riboflavin), B3 (niacin), B5 (pantothenic acid), B6, B7 (biotin), B12, and Folic acid, in any known form), any other known vitamins and minerals, chelated minerals or any combination thereof. It should be well understood that these are just examples and this patent shouldn't be interpreted as being limited to these examples.

Any formulation may contain one or more dietary supplements as defined and described by U.S. 21 C.F.R. part 111;

Any formulation of tablets may also include one or more super-disintegrant agents, including but not limited to croscarmellose sodium, crosslinked celluloses, crosslinked PVP, crosslinked starches, crosslinked alginic acid, calcium silicate. In particular, a combination of Polyplasdone XL and Calcium silicate was used for the examples given but any combination of such disintegrant can be used to achieve rapid disintegration time for tablets.

All formulations contain at least one binder including but not limited to: polyethylene glycols (PEG's) with high molecular weights (such as Carbowax 8000, Carbowax 6000, or Carbowax 4000 in powder or granulated forms), microcrystalline celluloses (MCC's), Carboxy methyl celluloses (CMC's), and mannitol and/or xylitol and/or erytrytol. Any binder may be spray dried, regular, granulated or fine powder or any other suitable grade.

Formulations also may contain an anti-foaming agent, for example Xiameter (silicones type-polysiloxines, methylpolysiloxines) in order to avoid foaming at the surface of water when the consumers will dissolve the tablet.

All formulations may include one or more lubricant. Lubricants include but are not limited to sodium benzoate, Leucine, instantized L-Leucine, medium chain tryglycerides (MCT) powder, magnesium stearate, stearic acid, ascorbyl palmitate salts, as well as other salts.

In some embodiments one or more lubricant may be used to coat the tablet, and more preferably a tablet is coated with a minimum amount of one or more lubricants. As an advantage of using less lubricant is an increase in disintegration times of the tablet. To minimize the amount the amount of lubricant used, a spray gun may be used to apply lubricant to a tablet while tablet press, or other means of forming the tablets, is running in order to minimize the amount of lubricant needed for the tablet tooling and to protect them.

All formulations may contain one or more acids. Acids may include but are not limited to malic acid, adipic acid, fumaric acid, tartaric acid, sodium tartrate, potassium tartrate, alpha keto glutaric acid, ascorbic acid, sodium ascorbate, potassium ascorbate, citric acid (anhydrous or hydrated), sodium citrate or potassium citrate salts. More preferably citric acid is anhydrous, direct compressible, coated with starch or PEG or uncoated.

Formulations may contain one or more carbonates. Carbonates may include: alkali carbonates, including but not limited to sodium carbonate, potassium carbonate and their bicarbonates forms (for example sodium sesquicarbonate); or non-alkali carbonates including but not limited to arginine bicarbonate, carbonate salt, lysine carbonate or lysine bicarbonate; or carbonates in any other known form.

Formulations may contain a natural or artificial sweetener (such as sucralose, sugars sugar alcohols, acesulfame potassium, monk fruit extract, stevia extracts and a natural or artificial flavor or N&A Favor.

In some embodiments, formulations may have a stoichiometric ratio of acid to carbonate between 5:1 and 1:10, and more preferably is a range between 1:1 to 1:10, a range between 3:2 and 2:3, or is approximately 5:1, 4:1, 3:1, 5:2, 2:1, 3:2, 4:3, 1:1, 3:4, 2:3, 1:2, 2:5, 1:3, 1:4, 1:5, 1:6, 1:7, 1:8, 1:9, or 1:10. In some embodiments the formulation, acids may be combined to react with the carbonate, and formulations may use an excess of the acid.

In some embodiments, the type of acid may affect performance of carbonation process, and in turn the ratio of acid to carbonate may affect the product. In general, higher ratios of acid to carbonate are preferable, as they yield faster reactions, and assure that the carbonate is completely reacted. 1:1 weight ratios of acid to total carbonate are common. However, highly reactive, highly soluble systems can use acid to carbonate ratios as low as 1:10.

Certain components of formulations according to embodiments of the present disclosure may have a dietary intake purpose. Several formulations are contemplated according to the embodiments of the present disclosure. While formulations are described below, it should be appreciated that other formulations may be provided without departing from the present disclosure. It is also being appreciated that different amounts of each component of the formulations may be included without departing from present disclosure.

There should be well understood the differences between this disclosure and the state of the art. Obtaining a granular powder material prior to tableting is part of the process in this disclosure, but not the purpose of this disclosure. It is well known that the terminology for an “granulate” refers to a “powder” and not for a “tablet”. There are significant differences on the size of such granulates powders and the actual tablet obtain by the present disclosure. Effervescent compositions are obtained by different techniques such as wet granulation, fluidized bed, spray-coating, direct compression on an industrial tablet press, and others. The finished product is obtained here is spheroidal and much bigger size than the granulated material. Thus, it should be appreciated that the granulated particle size is usually at the microns level, where the actual spheroidal shaped tablet obtained in present disclosure has a diameter of at least 0.30 inches, and more preferable 0.75 inches diameter.

The present disclosure describes a tablet and tooling used for tableting said tablet in a shape of a capsule with a first diameter of approximately 0.1-0.5 inches and a second diameter approximately 0.2-1.5 inches.

The present disclosure describes a tablet and tooling used for tableting said tablet in a shape of a capsule with a first diameter and a second diameter, wherein the first diameter and second diameter may independently be, approximately 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 inches.

The present disclosure describes a tablet and tooling used for tableting said tablet in a shape of a sphere where the diameter that may be approximately 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 inches.

More preferably, the present disclosure describes tooling used for tableting in a shape of a capsule with a first diameter of approximately 0.325 inch and a second diameter approximately 0.75 inch. The calculated total surface area for a cylindrical shape tablet that has the same diameter as the capsule shape tablet is approximately 0.66 inch² for an approximately 0.325 inch diameter and approximately 4.46 inch² for an approximately 0.75 inch diameter respectively calculate for a high of approximately 5 mm.

The total surface area for a capsule shape tablet with the same high on the cylindrical part and the same diameter and height as for the cylindrical shape is: 1.73 inch² for a 0.325 inch diameter and 7.07 inch² for a 0.75 inch diameter. It should be understood that the surface area is bigger in the spheroidal shape tablets allowing for obtaining fast dissolving tablets.

The spheroidal shape tablet consists in 2 half spheres top and bottom and the middle die is a flat edge cylindrical shape so that the tablet can form and not break/capping during compression.

The capsule shape tablet consists in 2 half oblong spheres top and bottom and the middle die is a flat edge cylindrical shape so that the tablet can form and not break/capping during compression.

It should be observed that the spheroidal shape tablet offer bigger surface area than the cylindrical shape tablet which results in uniform high speed reaction between the bicarbonates/carbonates and the organic acids type used for effervescent reaction;

The production of spheroidal shaped effervescent tablets requires special tooling and a good external lubricating system in place that will minimize the amount of lubricant used and reduce the ejection force require for such big tablets when running the machine at a full speed. In this disclosure, the tablets were made with internal and external lubrication with magnesium stearate or ascorbyl palmitate as external lubricants and also with carbowax and sodium benzoate when used as internal lubricants.

The hardness of the tablets described herein, may be between 5 kPa to 20 kPa in order to achieve comparable dissolution rates as obtained for a classic disk shape effervescent tablet when compared by weight. A special ejection spring was made and installed in a Stoke tablet press machine in order to avoid the tablets to cap on ejection.

The hardness of any tablet described herein is preferably 5, 6, 7, 8, 9, 10, 11, 12, 13, 14, 15, 16 17, 18, 19, or 20 kPa.

When manufacturing the tablets, the relative humidity in the production room should be maintained at no more than approximately 30%, and more preferably humidity is under 5%, 7.5%, 10%/o, 12.5%, 15%, 17.5%, 20%, 22.5%, or 25%.

It was surprisingly observed during the experiments that the tablet press can be operated after adjustments at the full speed and comparable production rate as for regular tablets.

Accordingly, overviews of exemplary formulations are provided below. Formulations described herein may contain any combination of individual components listed below at Tables 1, 2, 3, 4, 5, 6, 7, or 8.

Table 1 shows a typical Energy formula.

| Components | Function | Grams per tablet |
|---|---|---|
| Effersoda ™ | excipient | 1.882 |
| (surface treated | | |
| sodiumcarbonate/sodium | | |
| bicarbonate) | | |
| Citric acid anhydrous | excipient | 1.32 |
| Perlitol Flash | excipient | 0.625 |
| Polyplasdone XL | excipient | 0.05 |
| Calcium silicate | excipient | 0.01 |
| PEG 8000 | excipient | 0.5 |
| Sodium Benzoate | excipient | 0.0015 |
| Xiameter antifoam | excipient | 0.005 |
| Sucralose | sweetener | 0.0375 |
| Flavor Berry Mix | flavor | 0.0615 |
| Natural | | |
| Caffeine Anhydrous | dietary | 0.1 |
| | ingredient | |
| Alpha Yohimbine | dietary | 0.002 |
| | ingredient | |
| Huperzin A 1% | dietary | 0.001 |
| | ingredient | |
| Arginine Base | dietary | 0.5 |
| | ingredient | |
| Vinitrox (apple extract) | dietary | 0.05 |
| | ingredient | |

| | |
|---|---|
| Total per tablet | 5.1455 |

Table 2 shows a typical Pre-Workout formula with Creatine.

| Ingredient | Function | Grams/tablet |
|---|---|---|
| Sodium | excipient | 1.882 |
| Bicarbonate | | |
| Citric acid | excipient | 1.32 |
| anhydrous | | |
| Perlitol Flash | excipient | 0.625 |
| Polyplasdone XL | excipient | 0.05 |
| Calcium silicate | excipient | 0.01 |
| PEG 8000 | excipient | 0.5 |
| Sodium Benzoate | excipient | 0.0015 |
| Xiameter antifoam | excipient | 0.005 |
| Sucralose | sweetener | 0.0375 |
| Flavor Orange | flavor | 0.0615 |
| Buffered Creatine | dietary | 0.5 |
| | ingredient | |

| | |
|---|---|
| Total per tablet | 4.9925 |

Table 3 shows a typical Pre-Workout formula with Beta Alanine.

| Ingredient | Function | Grams/tablet |
|---|---|---|
| Sodium | excipient | 1.654 |
| Bicarbonate | | |
| Citric acid | excipient | 1.190 |
| anhydrous DC | | |
| Perlitol Flash | excipient | 0.650 |
| Polyplasdone | excipient | 0.050 |
| XL | | |
| Calcium | excipient | 0.010 |
| silicate | | |
| PEG 8000 | excipient | 0.550 |
| Sodium | excipient | 0.002 |
| Benzoate | | |
| Xiameter | excipient | 0.005 |
| antifoam | | |
| Sucralose | sweetener | 0.038 |
| berry flavor | flavor | 0.062 |
| Beta Alanine | dietary ingredient | 0.533 |
| FD&C Red 40 | colorant | 0.002 |
| FD&C Yellow 5 | colorant | 0.003 |

| | |
|---|---|
| Total per tablet | 4.748 |

Table 4 shows a typical Fat Burner/Weight Loss formula.

| Ingredient | Function | Grams/tablet |
|---|---|---|
| Sodium Bicarbonate | excipient | 1.654 |
| Citric acid anhydrous | excipient | 1.190 |
| DC | | |
| Perlitol Flash | excipient | 0.650 |
| Polyplasdone XL | excipient | 0.050 |
| Calcium silicate | excipient | 0.010 |
| PEG 8000 | excipient | 0.550 |
| Sodium Benzoate | excipient | 0.002 |
| Xiameter antifoam | excipient | 0.005 |
| Sucralose | sweetener | 0.038 |
| Orange Flavor | flavor | 0.062 |
| GBB EE HCL | dietary | 0.020 |
| (gamma | ingredient | |
| butyrobetaine ethyl | | |
| ester hydrochloride) | | |
| L Carnitine Fumarate | dietary | 0.500 |
| | ingredient | |
| Paradoxine (6- | dietary | 0.025 |
| Paradol) | ingredient | |
| Dihydrocapsiate | dietary | 0.003 |
| | ingredient | |
| FD&C Red 40 | colorant | 0.002 |
| FD&C Yellow 5 | colorant | 0.001 |

| | |
|---|---|
| Total per tablet | 4.763 |

Table 5 shows a typical Post-Workout formula.

| Ingredient | Function | Grams/tablet |
|---|---|---|
| Sodium | excipient | 1.882 |
| Bicarbonate | | |
| Citric add | excipient | 1.32 |
| anhydrous | | |
| Perlitol Flash | excipient | 0.625 |
| Polyplasdone XL | excipient | 0.05 |
| Calcium silicate | excipient | 0.01 |
| PEG 8000 | excipient | 0.5 |
| Sodium Benzoate | excipient | 0.0015 |
| Xiameter | excipient | 0.005 |
| antifoam | | |
| Sucralose | sweetener | 0.0375 |
| Flavor Orange | flavor | 0.0615 |
| BCAA (branch | dietary | 0.7 |
| chain amino | supplement | |
| acids) | | |

| | |
|---|---|
| Total per tablet | 5.1925 |

Table 6 shows another typical Pre-Workout formula.

| Ingredient | Function | Grams/tablet |
|---|---|---|
| Sodium Bicarbonate | excipient | 1.5 |
| Citric acid anhydrous | excipient | 1.32 |
| Perlitol Flash | excipient | 0.525 |
| Polyplasdone XL | excipient | 0.05 |
| Calcium silicate | excipient | 0.01 |
| PEG 8000 | excipient | 0.35 |
| Sodium Benzoate | excipient | 0.0015 |
| Xiameter antifoam | excipient | 0.005 |
| Sucralose | sweetener | 0.02 |
| Blue Raspberry Flavor | flavor | 0.0615 |
| Caffeine Anhydrous | dietary | 0.32 |
| | supplement | |
| Alpha Yohimbine | dietary | 0.002 |
| | supplement | |
| Nitrosigine | dietary | 0.75 |
| | supplement | |
| Blue Lake | colorant | 0.003 |
| Blue Dye #1 | colorant | 0.003 |
| Citrus aurantium 30% p- | dietary | 0.04 |
| Synephrine | supplement | |

| | |
|---|---|
| Total per tablet | 5.161 |

Table 7 shows a typical Male Enhancement formula.

| Ingredient | Function | Grams/tablet |
|---|---|---|
| Citric acid anhydrous | excipient | 1.32 |
| Perlitol Flash | excipient | 0.55 |
| Polyplasdone XL | excipient | 0.05 |
| Calcium silicate | excipient | 0.01 |
| PEG 8000 | excipient | 0.60 |
| Sodium Benzoate | excipient | 0.00 |
| Xiameter antifoam | excipient | 0.01 |
| Sucralose | sweetener | 0.04 |
| Flavor Berry Mix Natural | flavor | 0.06 |
| Nitrosigine (stabilized | Dietary | 0.5 |
| Arginine Inositol silicate) | supplement | |
| Yohimbine HCL | Dietary | 0.015 |
| | supplement | |
| GBB HCL | Dietary | 0.2 |
| (gammabutyrobetaine ethyl | supplement | |
| ester Hydrochloride) | | |

| | |
|---|---|
| Total per tablet | 4.5705 |

Table 8 shows a typical Fat Burner formula.

| | | |
|---|---|---|
| Sodium Bicarbonate | Function | 1.654 |
| Citric acid anhydrous | excipient | 1.190 |
| DC | | |
| Perlitol Flash | excipient | 0.650 |
| Polyplasdone XL | excipient | 0.050 |
| Calcium silicate | excipient | 0.010 |
| PEG 8000 | excipient | 0.550 |
| Sodium Benzoate | excipient | 0.002 |
| Xiameter antifoam | excipient | 0.005 |
| Sucralose | sweetener | 0.025 |
| Lemonade | flavor | 0.062 |
| GBB EE HCL | Dietary | 0.200 |
| | supplement | |
| Paradoxine 12.5% 6 | Dietary | 0.200 |
| paradol | supplement | |
| P-5-P(pyridoxine-5- | Dietary | 0.050 |
| phosphate) | supplement | |
| FD&C Red 40 | colorant | 0.002 |
| FD&C Yellow 5 | colorant | 0.003 |

| | |
|---|---|
| Total per tablet | 4.653 |

Table 9 shows of effective dosages of dietary ingredients, that can be included in formulations used in a speroidal tablet.

| Ingredient Name | Effective dosage |
|---|---|
| Creatine Monohydrate or Anhydrous | 5,000 mg/dose/day |
| Buffered Creatine | 1,500 mg/dose/day |
| Beta Alanine | 1500-3000 mg/dose/day |
| L Citrulline | 1,000 mg/dose/3 times daily |
| L-Arginine | 3-6 grams/dose/3 times daily |
| BCAA (branch chain amino acids) | 3-4 g/dose/up to 10 grams per |
| | day depending on the goal |
| L-Leucine | 2 g to 5 g acute dose/day |
| HMB (Hydroxy methylbutyric acid) | 1-3 g/dose/day |
| L-Carnitine | 500 mg to 2000 mg |
| Acetyl L Carnitine | 630-2,500 mg/dose/day |
| L-Carnitine-Fumarate | 1,000 mg/dose per day |
| L-Carnitine L-Tartrate | 1,000-4000 mg/dose/day |
| Glycine-Propionyl-L-Carnitine | 1000-4000 mg |
| Agmatine Sulfate | 1,300-2,670 mg/dose/day |
| Alpha yohimbine | 2 mg/dosage/day |
| GBB EE HCL (gamma butyrobetaine | 50-400 mg/dosage/day |
| ethyl ester hydrochloride) | |
| Yohimbine HCL | 8-20 mg/dosage/day |
| Hupezine A | 1 mg/dose/day |
| Nitrosigine ™(Stabilized Arginine | 750-1500 mg/dosage/day |
| Inositol Silicate) | |
| BHB Salts (betahydroxybutyrate sodium, | 6-12 g/day |
| potassium calcium or magnesium) | |
| Paradoxine ™ (6-paradol) | 50 mg-200 mg/dose/day |

Methods of Making

Wet granulations method or the “fusion method” are known processes to create tablets in the pharmaceutical industry can be applied to dietary supplements industry prior to tableting for achieving a porous partial reacted material ready to use for tableting. Making a spheroidal tablet with the fusion method may achieve faster dissolving times of the spheroidal tablets. For example, a spheroidal tablet made with fused material can incorporate 1%-30%, more preferably more than 30% of the supplements into a formulation than what. Prior to using the fusion method dietary supplements with a crystalline structure may be milled prior to this treatment to a particle size of 80-200 mesh in order to maximize the surface area of contact between the components.

Size of mesh, mesh particle size or mesh size as used herein can be construed according commonly understood use in the art, may describe the size of opening in a sieve or other tool, and may be used to classify a particle size of the powders more precisely according to the US Standardized Mesh System.

In some embodiments, micronized 80 to 200 mesh particle size dietary ingredients can be used when fusion method is required. For example, beta alanine has a crystalline structure and aspect. Beta alanine must be milled to the same range of particle size, 80 mesh to 200 mesh. In general, all the dietary components must be milled prior to performing the fusion.

An example of processes useful for making formulations and spheroidal tablets disclosed are below.

Step 1: Sift citric acid, sodium bicarbonate, and sodium carbonate. These salts can be replaced with other salts, including but not limited to potassium carbonate/potassium bicarbonate and or any other citrates, tartrates, ascorbates, adipates, malates, alpha keto glutarates salts and/or any hydrated forms of same (for example sodium carbonate decahydrate). The preferred ratio (acid: carbonate/bicarbonate) to make the effervescent reaction is 50:50, but is not limited to this ratio, with our without the dietary ingredients to pass through the #60 mesh size; this may be done on a stainless still vibratory sifting tower and in a room with low humidity, preferable to no more than 30% relative humidity (RH); the water of hydration from the hydrates salts used will partially hydrate the mixture and allow for obtaining a porous mass which is partial reacted and ready to use after drying for tableting

Step 2: Mix all components into a ribbon blender or a V shape blander or a double cone blender or any rotatory blender for about 3 to 5 minutes; while mixing spray purified water in amount of 2% or 2 g per Kg mixed powder weight. This step can be achieved on industrial scale by using fluidized bed equipment which can include also the drying step;

Step 3: Discharge the blend into a drying tray and dry the mix into an regular drying oven or a microwave industrial oven preheated at around 40-90 degree C., most preferable to 70-90 degree C. for a period of about 90 minutes; while drying turn up or move the material with the material so the drying is uniform. This material can be than use for making tablets.

After creating the formulation by a process described herein, or otherwise known in the art, a tablet can be created from that powder by using an industrial tablet press that is equipped with upper and lower die punches to make spheroidal tablets.

More particularly, a tablet may be made from any formulation described herein by 1) loading the mixture into a tablet press and operate tablet press to create a tablet; 2) adjusting the tablet press to achieve a weight and a hardness of the tablet, wherein said weight and said hardness are within a specified range; 3) load external lubricant solution onto the automatic sprayer and spray tablet press, in order to encase the resulting tablet with a thin layer of lubricant; and 4) releasing the tablet from the tablet press.

One embodiment includes tablets that are compressed in a spheroidal shape to achieve a hardness from 7 kPa to 20 kPa, and more preferable tablets have a hardness of 7.0 kPa, 7.5 kPa, 8.0 kPa, 8.5 kPa, 9.0 kPa, 9.5 kPa, 10.0 kPa, 10.1 kPa, 10.2 kPa, 10.3 kPa, 10.4 kPa, 10.5 kPa, 10.6 kPa, 10.7 kPa, 10.8 kPa, 10.9 kPa, 11.0 kPa, 11.1 kPa, 11.2 kPa, 11.3 kPa, 11.4 kPa, 11.5 kPa, 11.6 kPa, 11.7 kPa, 11.8 kPa, 11.9 kPa, 12.0 kPa, 12.1 kPa, 12.2 kPa, 12.3 kPa, 12.4 kPa, 12.5 kPa, 12.6 kPa, 12.7 kPa, 12.8 kPa, 12.9 kPa, 13.0 kPa, 13.1 kPa, 13.2 kPa, 13.3 kPa, 13.4 kPa, 13.5 kPa, 13.6 kPa, 13.7 kPa, 13.8 kPa, 13.9 kPa, 14.0 kPa, 14.1 kPa, 14.2 kPa, 14.3 kPa, 14.4 kPa, 14.5 kPa, 14.6 kPa, 14.7 kPa, 14.8 kPa, 14.9 kPa, 15.0 kPa, 15.5 kPa, 16.0 kPa, 16.5 kPa, 17.0 kPa, 17.5 kPa, 18.0 kPa, 18.5 kPa, 19.0 kPa, 19.5 kPa, 20.0 kPa.

Another embodiment includes tablets that are compressed in a spheroidal shape in order to be transportable without capping or breaking and be able to dissolve in water or another aqueous solution, at room temperature, approximately 19 to 23 C, in approximately 1 to 3 minutes, more preferably dissolving in approximately 0.5, 0.75, 1.0, 1.25, 1.5, 1.75, 2.0, 2.25, 2.5, 2.75, or 3.0 minutes.

Another embodiment includes tablets that are compressed in a spheroidal shape with a hardness that enables the tablets to be transportable without capping or breaking and also be able to dissolve in cold water or another cold aqueous solution, in approximately 1 to 5 minutes, and more preferably dissolving in 0.5, 0.75, 1.0, 1.25, 1.5, 1.75, 2.0, 2.25, 2.5, 2.75, 3.0, 3.25 3.5, 3.75, 4.0, 4.25, 4.5, 4.75, or 5 minutes. Cold water or cold aqueous solution may have a temperature between 1 and 18 C.

### EXPERIMENTAL RESULTS

The spheroidal shape allows good disintegration times in water at room temperature or cold water. The disintegration times of the spheroidal tablets are similar to the classic flat big surface area cylinder shaped effervescent tablets such as Alkaseltzer when calculate by weight and is able to incorporate more active components in particular any dietary supplement ingredient at the effective dosage. The spheroidal tablet will allow for obtaining comparable dissolution rates despite the fact that the spheroidal tablets weight is much higher.

Carbon dioxide evolve is equal to the final weight minus the initial weight minus the average weight of the tablet. It can be represented by this formula:

CO₂E=(W_{f}−Wᵢ)−A_{w}, wherein
CO₂E is the Carbon Dioxide Evolve;
W_{f} is the final Weight;
Wᵢ is the initial Weight; and
A_{w} is Average weight of the tablet.

Weight Variation of Optimized Batch.

Tested batches used weight variation of optimized batches. In order to analyze the uniform distribution of active ingredients in the final formulation parameters like weight variation. For weight variation test, ten tablets were taken and weighed individually.

Criteria to Pass Testing.

The following criteria were required to pass testing: 1) No more than 2 tablets differ by more than 5% relative to average weight; and 2) No tablet differs in weight by more than 10% of the average weight.

Carbon Dioxide Loss by Gravimetric Method.

Carbon dioxide loss by gravimetric method used to weight tablets. The tablet was accurately weighed and added to weighed quantity of approx. 100 g of water. The CO₂ gas evolved which led to the reduction in weight of total quantity. The final weight was noted and reduction in weight was calculated.

The calculated average of the carbon dioxide evolved was between 0.3 g and 0.4 g with an average of 0.4 g which is a good indication that the effervescent reaction occurred and is in direct correlation with the time that the tables will dissolve.

Determination of Change in pH:

The pH of demineralized water was noted initially, then the developed tablet was added to it and after completion of evolution of carbon dioxide, the pH of the fluid was again noted.

There was observed a slightly drop in the pH of the solutions between 1.1 and 2.6 units which means that there was enough acid added in excess of the stoichiometrical amount calculated to be needed for the effervescent reaction to occur and complete, so that the excess amount of acid will be used for the purpose of obtaining the desire good flavor and taste.

The following tables present results for weigh variation test using the techniques described above.

Table 1 shows results for a tablet made with a Pre-Workout formula with Beta Alanine.

| | | | | |
|---|---|---|---|---|
| Product Name: | Beta Alanine | Range Weight (g) | 4.3 | 5.3 |

| | | |
|---|---|---|
| Technician: | JVH | Lot# Test Batch |
| Equipment: | Mettler Toledo AG104 | Method: USP <2091> |

| Number of | Tablet | Pass/ | Done | | Check | |
|---|---|---|---|---|---|---|
| Tablets | Weight (g) | Fail | By: | Date: | By: | Date: |
| 1 | 4.8791 | Pass | | | | |
| 2 | 4.8130 | | | | | |
| 3 | 4.7867 | | | | | |
| 4 | 4.8643 | | | | | |
| 5 | 4.8783 | | | | | |
| 6 | 4.7712 | | | | | |
| 7 | 4.8152 | | | | | |
| 8 | 4.8460 | | | | | |
| 9 | 4.9123 | | | | | |
| 10 | 4.7823 | | | | | |
| Average Weight(g) | 4.8348 | | | | | |

Table 12 shows results for a tablet made with an Energy formula.

| | | | | |
|---|---|---|---|---|
| Product Name: | Energy | Range Weight (g) | 4.1 | 5.1 |

| | | |
|---|---|---|
| Technician: | JVH | Lot# Test Batch |
| Equipment: | Mettler Toledo AG104 | Method: USP <2091> |

| Number of | Tablet | Pass/ | Done | | Check | |
|---|---|---|---|---|---|---|
| Tablets | Weight (g) | Fail | By: | Date: | By: | Date: |
| 1 | 4.5714 | Pass | | | | |
| 2 | 4.5561 | | | | | |
| 3 | 4.5419 | | | | | |
| 4 | 4.5885 | | | | | |
| 5 | 4.5927 | | | | | |
| 6 | 4.6750 | | | | | |
| 7 | 4.6516 | | | | | |
| 8 | 4.6232 | | | | | |
| 9 | 4.5667 | | | | | |
| 10 | 4.6884 | | | | | |
| Average Weight(g) | 4.6056 | | | | | |

Table 13 shows results for a tablet made with a Fat Burner formula.

| | | | | |
|---|---|---|---|---|
| Product Name: | Fat Burner | Range Weight (g) | 3.8 | 4.8 |

| | | |
|---|---|---|
| Technician: | JVH | Lot# Test Batch |
| Equipment: | Mettler Toledo AG104 | Method: USP <2091> |

| Number of | Tablet | Pass/ | Done | | Check | |
|---|---|---|---|---|---|---|
| Tablets | Weight (g) | Fail | By: | Date: | By: | Date: |
| 1 | 4.3710 | Pass | | | | |
| 2 | 4.4669 | | | | | |
| 3 | 4.4520 | | | | | |
| 4 | 4.3805 | | | | | |
| 5 | 4.3519 | | | | | |
| 6 | 4.3699 | | | | | |
| 7 | 4.3685 | | | | | |
| 8 | 4.3446 | | | | | |
| 9 | 4.3510 | | | | | |
| 10 | 4.3997 | | | | | |
| Average Weight(g) | 4.3856 | | | | | |

Table 14 shows results for a tablet made with a Male Enhancement formula.

| | | | | |
|---|---|---|---|---|
| Product Name: | Male Enhancement | Range Weight (g) | 4.5 | 5.5 |

| | | |
|---|---|---|
| Technician: | JVH | Lot# Test Batch |
| Equipment: | Mettler Toledo AG104 | Method: USP <2091> |

| Number of | Tablet | Pass/ | Done | | Check | |
|---|---|---|---|---|---|---|
| Tablets | Weight (g) | Fail | By: | Date: | By: | Date: |
| 1 | 4.9860 | Pass | | | | |
| 2 | 5.0722 | | | | | |
| 3 | 4.9906 | | | | | |
| 4 | 4.9931 | | | | | |
| 5 | 5.5360 | | | | | |
| 6 | 4.9521 | | | | | |
| 7 | 4.9837 | | | | | |
| 8 | 4.9820 | | | | | |
| 9 | 5.0235 | | | | | |
| 10 | 5.0236 | | | | | |
| Average Weight(g) | 5.0543 | | | | | |

A number of embodiments have been described herein. Nevertheless, it will be understood that various modifications may be made without departing from the spirit and scope of the claimed invention. In addition, any logic flows depicted in Figures, illustrated in examples, or are otherwise explained in any level of detail, are not limited to the details as described or shown, and do not require the particular order shown, or sequential order, to achieve desirable results.

In addition, other steps may be provided, or steps may be eliminated, from the described flows, methods or processes, and other components may be added to, or removed from, the described systems. Accordingly, other embodiments are within the scope of the following claims.

It may be appreciated that the methods, processes and formulations disclosed herein may be embodied in a machine-readable medium and/or a machine accessible medium compatible with a data processing system (e.g., a computer system), and/or may be performed in any order.

The modules in the Figures may be shown as distinct and communicating with only a few specific examples, and not others. The information from the figures may be merged with each other, may show overlapping functions, and may communicate with other information not shown to be connected in the Figures. Accordingly, the specification and/or drawings may be regarded in an illustrative rather than a restrictive sense.

1. A method of making an effervescent spheroidal tablet by the steps of:
a. Sifting one or more acids, one or more carbonates, and one or more dietary ingredients with a stainless steel sieve to create a mixture of components;
b. Mixing the mixture in a blender, and concurrently spraying mixing spray onto the mixture, until the mixture and mixing spray are homogenized;
c. Discharging the mixture onto a drying tray;
d. Drying the mixture in a preheated oven for a period of approximately 90 minutes, concurrently agitating the mixture;
e. Optionally allowing the mixture to cool to room temperature; and
f. Loading the mixture into a tablet press and operate tablet press to create a tablet.

2. The method of claim 1, wherein step a) further includes sifting one or more super-disintegrant agents, one or more binders, one or more lubricants, and one or more sweeteners to include in the mixture of components.

3. The method of claim 1, wherein the one or more dietary ingredient is selected from a group consisting of caffeine anhydrous, tea extracts, purine alkaloids, theacrine, methyl liberine, 1,3,7 trimethyluric acid, 1,3,7 trimethyluric acid sodium salts, yohimbine, alpha yohimbine, yohimbine hydrochloride, huperzin A, vinitrox, creatine, creatine salts, buffered creatine, beta alanine, gamma butyrobetaine ethyl ester, gamma butyrobetaine ethyl ester hydrochloride, gammabutyrobetaine ethyl ester, medium chain triglycerides, carnitine, L-carnitine, fumarate, tartrate, paradoxine, dihydrocapsiate, branch chain amino acids, any mixture of one or more amino acids, leucine, isoleucine, valine, arginine base, alpha ketoglutarate hydrochloride, agmatine, agmatine salts, nitrosigine, B vitamins, and any pharmaceutical salts thereof.

4. The method of claim 1, wherein the blender is selected from group consisting of a ribbon blender, V-shaped blender, double-cone blender, any medium size, pilot size or any rotary industrial blender, spray drying blenders and fluidized bed equipment.

5. The method of claim 1, wherein stoichiometric ratio of the one or more acids to the one or more carbonates is between 1:3 and 1:10.

6. The method of claim 1, wherein stoichiometric ratio of the one or more acids to the one or more carbonates, derived the sodium bicarbonate and the sodium carbonate, allows the acid to quench the carbonate in an effervescent reaction, and after said effervescent reaction having an excess of the acid in an amount to achieve good tasting drinks after the tablet is dissolved in water completely.

7. The method of claim 1, wherein the stainless steel sieve has a mesh size between 20 and 200.

8. The method of claim 7, wherein the mesh size is between 40 and 80 mesh.

9. The method of claim 1, wherein the method is made in an environment, and said environment has a relative humidity of less than 30%.

10. The method of claim 9, wherein the environment has a relative humidity between 10 and 20%.

11. The method of claim 1, wherein the mixing spray is comprised of water and the solid powder mix, and said mixing spray is 0.1% to 2% by weight of the total solid mix, wherein solid powder mix represent all the components in the formulation that were prior mixed or just the acid/bicarbonate/carbonate mixture);

12. The method of claim 11, wherein the mixing spray is 0.5% to 1% by weight powder mix, wherein solid powder mix represent all the components in the formulation that were prior mixed or just the acid/bicarbonate/carbonate mixture);

13. The method of claim 1, wherein the preheated oven is between 40 and 90 C.

14. The method of claim 14, where in the preheated oven is between 70 and 90 C.

15. A spheroidal shaped effervescent tablet having a diameter between 0.1″ to 3″, wherein said tablet comprises one or more dietary ingredients, one or more super-disintegrant agents, one or more binder, one or more lubricant, one or more acids, one or more carbonates, and one or more sweeteners, and wherein said tablet disintegrates as quickly as a disc shaped containing the same amount of active ingredients.

16. A tablet of claim 15, wherein the spheroidal tablet is shaped like a capsule, having a first diameter between 0.25″ to 0.40″ and a second elongated diameter between 0.5″ and 1″.

17. A tablet of claim 15, wherein the one or more dietary ingredients is selected from any dietary supplement described in U.S. 21 C.F.R. part 111.

18. A tablet of claim 15, wherein the one or more dietary ingredient is selected from a group consisting of caffeine anhydrous, tea extracts, purine alkaloids, theacrine, methyl liberine, 1,3,7 trimethyluric acid, 1,3,7 trimethyluric acid sodium salts, yohimbine, alpha yohimbine, yohimbine hydrochloride, huperzin A, vinitrox, creatine, creatine salts, buffered creatine, beta alanine, gamma butyrobetaine ethyl ester, gamma butyrobetaine ethyl ester hydrochloride, gammabutyrobetaine ethyl ester, medium chain triglycerides, carnitine, L-carnitine, fumarate, tartrate, paradoxine, dihydrocapsiate, branch chain amino acids, any mixture of one or more amino acids, leucine, isoleucine, valine, arginine base, alpha ketoglutarate hydrochloride, agmatine, agmatine salts, nitrosigine, B vitamins, and any pharmaceutical salts thereof.
